SEQUENCE LISTING (6 sequences)

SEQ ID NO: 1 - DNA, 20 bp, synthetic construct
  misc_feature 1..20: note = synthesized
  source 1..20: mol_type = other DNA
accgaagagctaccagacga

SEQ ID NO: 2 - DNA, 20 bp, synthetic construct
  misc_feature 1..20: note = synthesized
  source 1..20: mol_type = other DNA
ctgcgtagaagccttttggc

SEQ ID NO: 3 - DNA, 46 bp, synthetic construct
  misc_feature 1..46: note = synthesized
  source 1..46: mol_type = other DNA
tccagcttctggcccagttcctttttattcgtggtggtgacggtaa

SEQ ID NO: 4 - DNA, 50 bp, synthetic construct
  misc_feature 1..50: note = synthesized
  source 1..50: mol_type = other DNA
tatgggttgcaactgagggagccttttttcattgttagcaggattgcggg

SEQ ID NO: 5 - DNA, 23 bp, synthetic construct
  misc_feature 1..23: note = synthesized
  source 1..23: mol_type = other DNA
accatcttggactgagatctttc

SEQ ID NO: 6 - DNA, 23 bp, synthetic construct
  misc_feature 1..23: note = synthesized
  source 1..23: mol_type = other DNA
tacaccaaaagatcacattggca

(19) United States
(12) Patent Application Publication
(10) Pub. No.: US 2023/0235312 A1
(43) Pub. Date: Jul. 27, 2023

(54) DETECTION METHOD USING LATERAL-FLOW PAPER CHIP CAPABLE OF MULTI-NUCLEIC ACID COLORIMETRIC DETECTION WITH ONE-STEP

(71) Applicant: AItheNutriGene Co., Seongnam-si (KR)

(72) Inventor: Jong Chul KIM, Seongnam-si (KR)

(21) Appl. No.: 17/863,306

(22) Filed: Jul. 12, 2022

(30) Foreign Application Priority Data
Jul. 21, 2021 (KR) 10-2021-0095317

(51) Int. Cl. C12N 15/10; C12Q 1/6806; C12Q 1/6844; B01L 7/00

(52) U.S. Cl. CPC C12N 15/1006; B01L 7/52; C12Q 1/6806; C12Q 1/6844; B01L 2200/10; C12Q 2563/107

(57) ABSTRACT

The present disclosure relates to a structure capable of simultaneously purifying and detecting a nucleic acid by directly applying a sample, and more particularly, to a structure capable of performing sample preparation, loop-mediated isothermal amplification, detection and analysis steps on a single chip by applying lab-on-paper technology, and capable of finally determining whether the disease or bacterial is infected by moving the sample in a lateral flow method.

### CROSS-REFERENCE TO RELATED APPLICATIONS

This application claims the priority of the Korean Patent Applications NO 10-2021-0095317 filed on Jul. 21, 2021, in the Korean Intellectual Property Office, the disclosure of which is incorporated herein by reference.

### TECHNICAL FIELD

The present disclosure relates to a structure capable of simultaneously purifying and detecting a nucleic acid by directly applying a sample without purifying the nucleic acid from the sample, and more particularly, to a structure having a high detection sensitivity in spite of directly applying a sample without purifying a nucleic acid from the sample based on an integrated system of lateral fluidity and capable of easily and quickly diagnosing a plurality of diseases by simultaneously detecting a plurality of target nucleic acids.

### BACKGROUND ART

As a level of medical service increases and diagnostic devices develop, a technology capable of quickly and conveniently measuring infectious microorganisms in daily life has been developed in order to effectively deal with the infectious microorganisms that threaten human health, such as new viruses, super bacteria, tuberculosis, and food poisoning bacteria. Molecular diagnosis capable of measuring the infectious microorganisms often requires a diagnostic method with excellent sensitivity and specificity, special equipment or reagents, or involves a complex process. An immunodiagnostic method is easy to reproduce with a kit, fast and simple, but has a problem of poor measurement sensitivity.

Currently, a diagnostic method using real-time PCR is known as the fastest and most sensitive diagnostic method, and diagnosis is usually possible within 8 hours. Currently, molecular diagnostic methods are rapidly developing along with the development of PCR technology and microchannel technology, and products that may be detected within 60 minutes, such as Alere™ I from Alere or Cobas Influenza from Roche, are also commercially available. However, in a case of a methodology capable of rapid molecular diagnosis, expensive analysis equipment or a high test cost is required, and several steps are required to implement the entire molecular diagnosis process, and thus there are still limitations in on-site diagnosis.

Currently, the common molecular diagnostic method uses real-time PCR, which is the most common due to its speed, but it is difficult to use easily because it requires large and expensive equipment for on-site diagnosis or primary and secondary hospitals. Molecular diagnosis mainly requires three steps: sample preparation, nucleic acid amplification, and detection. Although the nucleic acid amplification and detection may be reproduced simultaneously by real-time PCR equipment, the problem of sample preparation still remains.

On the other hand, Lab-on-paper technology refers to a technology based on an integrated system that allows sample preparation, loop-mediated isothermal amplification, detection, and analysis steps to be performed on a single chip. The Lab-on-paper technology has the advantage of not being limited to places such as detection sites because all reactions may be automated and quickly performed with a small paper and a chip structure mounted on the paper.

### DISCLOSURE

### Technical Problem

One aspect provides a multi-nucleic acid detection structure, including: a sample pad for receiving a biological sample; a first connection pad disposed on the sample pad and connecting the sample pad and a reaction pad; a reaction pad disposed below the first connection pad, including a primer capable of specifically binding to a target nucleic acid and a reagent for loop-mediated isothermal amplification (LAMP), and in which loop-mediated isothermal amplification occurs; a blocking pad disposed on the reaction pad and configured to maintain a reaction temperature and block evaporation of the sample; a second connection pad disposed on the reaction pad and having gold nanoparticles fixed thereon; a detection pad disposed below the second connection pad and configured to obtain a target nucleic acid amplified from a loop-mediated isothermal amplification reactant bound to the gold nanoparticles; an absorbent pad disposed laterally of the detection pad and absorbing the remaining sample; and a heating pad disposed below the sample pad, the reaction pad, and the second connection pad.

Another aspect provides a multi-nucleic acid detection structure in which the sample pad and the first connection pad; the reaction pad; the second connection pad; the detection pad; and the absorbent pad are sequentially disposed laterally at least in partial contact with each other.

Another aspect provides a kit for diagnosing a disease or bacterial infection, including the multi-nucleic acid detection structure.

Another aspect provides a method of providing information for diagnosing a disease or bacterial infection by using the multi-nucleic acid detection structure.

### Technical Solution

Terminologies used herein are to mention only a specific exemplary embodiment, and are not intended to limit the present disclosure. Singular forms used herein include plural forms as long as phrases do not clearly indicate an opposite meaning. A term “including” used in the present specification concretely indicates specific properties, regions, integer numbers, steps, operations, elements, and/or components, and is not to exclude presence or addition of other specific properties, regions, integer numbers, steps, operations, elements, components, and/or a group thereof.

All terms including technical terms and scientific terms used herein have the same meaning as the meaning generally understood by those skilled in the art to which the present disclosure pertains unless defined otherwise. Terms defined in a generally used dictionary are additionally interpreted as having the meaning matched to the related art document and the currently disclosed contents and are not interpreted as ideal or formal meaning unless defined. Hereinafter, a multi-nucleic acid detection structure according to a preferred exemplary embodiment of the present disclosure will be described with reference to the accompanying drawings. Terminologies used herein are to mention only a specific exemplary embodiment, and are not intended to limit the present disclosure. Singular forms used herein include plural forms as long as phrases do not clearly indicate an opposite meaning. A term “including” used in the present specification concretely indicates specific properties, regions, integer numbers, steps, operations, elements, and/or components, and is not to exclude presence or addition of other specific properties, regions, integer numbers, steps, operations, elements, components, and/or a group thereof.

Hereinafter, a multi-nucleic acid detection structure according to a preferred exemplary embodiment of the present disclosure will be described with reference to the accompanying drawings

A disease or bacterial infection diagnosis system according to the present disclosure is based on Lab-on-paper chip technology, and if the disease or bacterial diagnosis system according to the present disclosure is used, a nucleic acid material may be purified while moving to the reaction pad even without separate nucleic acid purification and may be directly applied to the amplification, and multiple target nucleic acids may be simultaneously detected and related diseases may be diagnosed by applying a single sample. To realize this, a nucleic acid detection structure according to the present disclosure includes a sample pad 120, a first connection pad 131, a reaction pad 140, a heating pad 141, a blocking pad 142, a second connection pad 150, a detection pad 160, an absorbent pad 170, and a housing 110 as components.

Hereinafter, each component will be described in detail.

### Sample Pad

The sample pad 120 accommodates a sample containing a nucleic acid material. The sample is isolated from the human body and includes, but is not limited to, blood, serum, plasma, saliva, sweat, urine, cell culture solution, tissue suspension, etc. The sample may be mixed with a cell lysis buffer, and if necessary, may be further purified by commonly known methods such as centrifugation, filtration, and precipitation, after mixing with the cell lysis buffer. Preferably, after the sample is mixed with a cell lysis buffer, purifying for application to the sample pad outside the nucleic acid detection structure is not included.

The cell lysis buffer may contain tris(hydroxymethyl) aminomethane in an amount of 5 mM to 80 mM, 5 mM to 50 mM, or 10 mM to 50 mM. Tris may specifically be Tris-HCl, and may reduce a rapid pH change as a buffering agent in the cell lysis buffer.

The cell lysis buffer may have a pH of 8.0 to 9.0. If the pH of the cell lysis butter is less than 8.0, the nucleic acid material may have reduced stability or a reduced movement speed.

The cell lysis buffer may contain potassium chloride (KCl) in an amount of 5 mM to 50 mM, 5 mM to 40 mM, or 10 mM to 20 mM. A high concentration of potassium chloride exceeding 50 mM is helpful for cell lysis, but reduces an aqueous solubility of an eluted nucleic acid material. Thus, it may be necessary to add a large amount of the addition buffer, or the time taken to move to the reaction pad may be increased. On the other hand, if the concentration of potassium chloride is less than 5 mM, cells may not be lysed properly.

The cell lysis buffer may contain magnesium sulfate (MgSO₄) in an amount of 1 mM to 30 mM, 1 mM to 20 mM, or 2 mM to 16 mM. If magnesium sulfate is contained in an appropriate amount, it was confirmed that the stability and movement speed of the nucleic acid material are increased, and it does not affect the viscosity, so it does not interfere with a flow of fluid, which is advantageous for pre-treatment of samples for analysis of paper chips.

The cell lysis buffer may contain ammonium sulfate ((NH₄)₂SO₄) in an amount of 5 mM to 50 mM, 5 mM to 40 mM, or 10 mM to 20 mM. A high concentration of ammonium sulfate exceeding 50 mM may precipitate cell lysates, and if ammonium sulfate is less than 5 mM, pH may become unstable.

The cell lysis buffer may contain 0.01 mg/ml to 0.1 mg/ml, or from 0.03 mg/ml to 0.07 mg/ml of protease. The protease decomposes high molecular weight proteins so that the nucleic acid does not block pores of a substrate or paper as a movement path by the high molecular weight proteins, and inhibits RNase and DNase activities to increase stability of the nucleic acid material. The protease may be proteinase K.

The surfactant may be TritonX-100 or Tween20 (Polysorbate 20), and may be contained in an amount of 0.01 w/w% to 0.2 w/w%, preferably 0.05 w/w% to 0.1 w/w%, based on the weight of the cell lysis buffer.

The cell lysis buffer may be used in a ratio of 1:1 to the volume of the sample.

The cell lysis buffer may not contain glycerol. Glycerol is sometimes added to prevent protein precipitation, but glycerol may increase viscosity to reduce fluidity of cell lysates, and may interfere with the movement of the nucleic acid material.

The cell lysis buffer may not contain a reducing agent. Reducing agents, for example, dithiothreitol (DTT), mercaptoethanol, etc., help to denature proteins and increase solubility of the cell lysates in water, but if the reducing agents are present in solution flowing into the paper chip, they may interfere with fluorescence or detection reactions.

The nucleic acid detection structure includes a heating pad 141 disposed below the sample pad and heating the sample pad. In order to efficiently dissolve the sample by the cell lysis buffer, the heating pad is heated at a temperature of 60 to 80° C. for 1 to 5 minutes, preferably for 5 minutes to promote dissolution of the sample containing virus and epithelial cells in the sample pad.

When a cell lysis composition is applied, the sample pad 120 made of a polysulfone membrane (e.g., Vivid GF) or a nitrocellulose membrane promotes cell lysis and makes nucleic acid more easily detected. The polysulfone membrane and the nitrocellulose membrane may have a structure in which two or more are stacked. The polysulfone membrane may be asymmetric, and the polysulfone membrane and the nitrocellulose membrane may be made of a porous material having pores of 0.5 µm to 1 µm. It is advantageous that the pores are rather large, and many biological samples have a viscosity, and in particular, when the sample is treated with the cell lysis composition, the viscosity may be increased significantly as the nucleic acid material and protein are eluted out of the cell. Therefore, it is preferable that the pore of the sample pad has a size suitable for rapidly absorbing the sample.

The nucleic acid may be more easily detected in a lateral flow manner using the cell lysis buffer. The term “lateral flow manner” refers to a manner of allowing a sample to flow from an applied point to a target point by a capillary phenomenon or a diffusion phenomenon in a horizontal direction without using gravity. The cell lysates contain a large amount of hydrolytic enzymes capable of degrading the nucleic acid material. Thus, if the retention time in the movement path is long, the yield of the nucleic acid material may be reduced. Therefore, in order to be applied to a nucleic acid detection structure in a lateral flow method, a flow rate should be good, and the cell lysates should not be precipitated to block the movement path during sample movement. In addition, it should not form a salt with the nucleic acid material to reduce precipitation or movement speed. Even if a composition of the cell lysis buffer according to the present disclosure does not contain glycerol or a reducing agent, the cell lysates or the nucleic acid materials are not precipitated, and the nucleic acid materials may be moved laterally to the reaction pad in a high yield.

### First Connection Pad

The first connection pad is a structure disposed on the sample pad and having a relatively narrow width compared to the sample pad, and connects the sample pad and the reaction pad to each other.

The first connection pad is made of a cellulose film, and may have pores of 0.005 µm to 0.015 µm.

### Reaction Pad

The reaction pad is a component corresponding to the paper chip in the Lab-on-paper, and a loop-mediated isothermal amplification reagent including dNTP, DNA polymerase, reverse transcriptase, fluorescent marker, loop-mediated isothermal amplification buffer, etc., for amplification is fixed to the reaction pad. Thus, if the solution containing the nucleic acid material is permeated and wetted into the reaction pad by the addition buffer applied to the sample pad, and the contact material of the loop-mediated isothermal amplification reagent and the sample moves to the reaction pad, and the temperature is increased to 60 to 70° C. by the heating pad below the reaction pad, a loop-mediated isothermal amplification or a reverse transcription loop-mediated isothermal amplification occurs.

The loop-mediated isothermal amplification reagent may specifically include dNTP (1.4 mM, dATP, dCTP, dGTP and dTTP), loop-mediated isothermal amplification buffer (1x, 20 mM Tris-HCl, 10 mM (NH₄)₂SO₄, 50 mM KCl, 2 mM MgSO₄, and 0.1% Tween- 20, pH 7.5), and Bst 3.0 DNA polymerase (320 U/ml), which may be mixed in the reaction pad and dried, or applied in a powder type on the surface of the reaction pad, heated, for example, in an oven at about 35 to 40° C. for about 30 minutes and fixed.

The reaction pad 140 may partially contact the first connection pad and the second connection pad, and may be disposed below and laterally of the first connection pad. A heating pad 141 may be disposed below the reaction pad 140 to perform heating to a temperature at which loop-mediated isothermal amplification can occur. The heating pad may include a heating wire or a heating plate for heating. The heating may be performed at 60 to 70° C., preferably 60 to 65° C. for 20 minutes to 1 hour, preferably 20 minutes to 30 minutes.

In order to increase the efficiency of the loop-mediated isothermal amplification, a blocking pad 142 may be disposed on the reaction pad to play a blocking role. It is possible to increase the efficiency of the reaction by maintaining the loop-mediated isothermal amplification temperature and blocking the evaporation of reagents by means of a blocking pad laminated with a series of arranged pads. The blocking pad may be a non-porous membrane or a structure capable of blocking the reaction pad from external air.

There are a plurality of wells in the reaction pad, and as a primer set for loop-mediated isothermal amplificationa forward and backward inner primers (FIP and BIP, 1.6 µM), loop primers (forward loop primer (FL) and backward loop primer (BL), 0.4 µM), and outer primers (F3 and B3, 0.2 µM) may be immobilized in each well. The concentration of primers is a concentration with respect to the volume of the well, and the concentration of a primer set in the well may be changed while maintaining a concentration ratio between each primer. Since the well is present in the reaction pad, the loop-mediated isothermal amplification may occur more intensively. Specifically, the well may have a hydrogel layer formed at the bottom, and the primer set may be fixed to the hydrogel layer. For intensive amplification of a specific target nucleic acid, a set of primers specifically binding to different target nucleic acids may be immobilized in each well.

The hydrogel layer containing the primer may be formed, for example, by the following method. 20% v/v of UV-photocrosslinkable poly(ethylene glycol) diacrylate (PEGDA, Sigma-Aldrich, MW700), 40% v/v of poly(ethylene glycol) (PEG, Sigma-Aldrich, MW600), 5% v/v of 2-hydroxy-2-methylpropiophenone (Sigma-Aldrich) as a photoinitiator, and 35% of buffer (PBS buffer, pH 7.5) are mixed based on the total volume of the hydrogel solution, and a primer set is mixed thereto to prepare a hydrogel solution. The poly(ethylene glycol) is preferably included in order to increase porosity of the hydrogel microparticles. Then, the hydrogel solution is applied to the inner surface of each well of the reaction pad and exposed to UV for 1 minute (360 nm wavelength, 35 mJ/cm²) to form a hydrogel coating layer. Since The hydrogel layer has pores and binds to the primers in the hydrogel layer, such that the amplification may occur intensively within the pores.

In the primer set, any one of the forward and backward primers may be labeled with one or more fluorescent markers selected from the group consisting of Cy3, Cy5, TAMRA, TEX, TYE, HEX, FAM, TET, JOE, MAX, ROX, VIC, Cy3.5, Texas Red, Cy5.5, TYE, BHQ, Iowa Black RQ, and IRDye. The fluorescent marker may be differently labeled for each target nucleic acid in order to independently detect the target nucleic acid.

In the primer set, the other one of the forward and backward primers may be biotin-bound. Since biotin may bind to streptavidin, it exists in a form bound to the amplified target nucleic acid, passes through the second connection pad, binds to streptavidin on the surface of the gold particle, and allows the detection result to be visible when captured by the detection pad. Biotin may be designed to be present at a position opposite to the detector in the amplified target nucleic acid, and for example, when a detector is bound to the 5′ end of the forward primer, biotin may be bound to the 5′ end of the backward primer.

The reaction pad is made of a material of a cellulose acetate membrane, and may have a pore size of 0.001 µm to 0.005 µm, preferably 0.005 µm so that the sample and the addition buffer are allowed to flow freely while the nucleic acid remains therein to sufficiently perform a loop-mediated isothermal amplification.

The reaction pad may include 40 mM to 50 mM of sucrose, 0.001 to 0.01% of Triton X-100, and 0.1w/w% to 0.3w/w% of glycerol. This may increase a storage stability of the loop-mediated isothermal amplification reagent, primer set, etc. when exposed to moisture or oxygen. “Storage stability” may mean that they may be stored for 3 weeks or more without degradation product or by-products at 25° C. to 30° C.

### Second Connection Pad

The second connection pad 150 may partially contact the reaction pad and be disposed on and laterally of the reaction pad. The second connection pad 150 includes gold nanoparticles, and the gold nanoparticles bind to the nucleic acid amplified in the reaction pad and move to the detection pad. The gold nanoparticles may preferably have streptavidin immobilized on the surface. The second connection pad is made of a porous material such as cotton, wool, paper, nitrocellulose, glass fiber, polysulfone, polyacrylic, polynitrile, polypiperazine, polyamide, polyethersulfone, polyvinylidene fluoride, polyethyleneimine, polydimethylsiloxane or a mixture thereof so that the amplified nucleic acid may easily move to the detection pad, and may have a pore size of 0.01 µm to 0.05 µm, preferably, 0.05 µm.

The second connection pad may be made of a cellulose material, and the second connection pad at a portion in contact with the reaction pad may be coated with low-melting agarose or wax. When the pad is coated, the movement of the loop-mediated isothermal amplification reactant of the reaction pad is blocked, and then the coating melts at the time of heating the second connection pad, and the loop-mediated isothermal amplification reactant moves laterally again, thereby preventing the loss of unreacted dielectric material in the sample.

A heating pad 141 for heating may be disposed below the second connection pad. When the addition buffer is added at an end point in time of the loop-mediated isothermal amplification, the resultant of the loop-mediated isothermal amplification is easily moved from the reaction pad to the detection pad. The heating may be performed at 60 to 80° C. for 1 minute to 5 minutes, preferably for 2 minutes.

### Detection Pad

The detection pad 160 may partially contact the second connection pad and be disposed below and laterally of the second connection pad. A receptor capable of binding to a detector is fixed to the detection pad 160. The receptor may be an antibody, protein, or fragment thereof capable of specifically binding to the detector.

The detection pad may include a plurality of detection zones, and the detection zones may be divided into lines or wells. Each receptor may be independently fixed in each detection zone, for example, after making a detection zone by stamping with an ink containing a polyethylene phthalate component, the detection zone may be stamped again with ink containing a receptor or, in the case of a well, a solution containing a receptor may be applied, and 1-ethyl-3-[3-dimethylaminopropyl] carbodiimide (EDC) or N-hydroxysulfosuccinimide (NHS) may be applied. Dozens to hundreds of detection zones or wells are formed in one reaction pad, and target nucleic acids as many as the number of detection zones formed by applying a single sample may be detected. FIG. 5 is for explaining in detail a structure of the detection zone formed on the reaction pad, and is not meant to limit the number of the detection zones

The detection pad is made of nitrocellulose, and may have a pore size of 0.001 µm to 0.005 µm, preferably 0.005 µm, so that the sample may move laterally to the absorbent pad.

### Heating Pad

A heating pad 141 may be disposed below the sample pad, the reaction pad, and the second connection pad. The heating pad is a metal plate having thermal conductivity, and may be made of a material such as iron, stainless steel, aluminum, silver, or copper. The heating pad may be connected with a heating wire, and each heating wire may be independently connected to the heating pad below the sample pad, the reaction pad, and the second connection pad to control a heated pad.

The sample pad, the first connection pad, the reaction pad, the second connection pad, the detection pad, and the absorbent pad may have housings 110 at upper and lower portions so that the sample or buffer is not lost, and the entire structure may be surrounded by a support body that may be defined as a housing or a case made of a non-porous material.

The absorbent pad serves to block a reverse flow by absorbing samples, buffers, etc. and to allow lateral fluidity to be induced. The absorbent pad is made of a porous material, and may be made of cotton, wool, paper, nitrocellulose, cellulose acetate, glass fiber, polysulfone, polyacrylic, polynitrile, polypiperazine, polyamide, polyethersulfone, polyvinylidene fluoride, polyethyleneimine, polydimethylsiloxane, or a mixture thereof. The absorbent pad may be preferably a glass fiber and may have a pore size of 0.1 to 0.5 µm.

The structure according to the present disclosure may purify the nucleic acid material by filtering the cell lysates in addition to the nucleic acid material while the sample reaches the absorbent pad 170 due to the arrangement and characteristics of each component of the structure, and may implement lateral fluidity in which the movement direction of the sample is parallel to the ground. In cells, nucleic acids exist in a form to which proteins are bound such as histones, polymerases, nucleases, and transcription factors. Many proteins lose their binding to nucleic acids by surfactants such as cell lysis compositions, but by the time the cell lysates reach the reaction pad by distilled water or additional buffer added to allow the cell lysates to move to the reaction pad, the surfactant is diluted so that the external environment may restore the charge of the protein again. In this case, the protein may bind to the nucleic acid material again to reduce the contact area with the polymerase or interfere with the amplification. Therefore, it is preferable that most of the cell components capable of binding to nucleic acids are purified and removed in the reaction pad.

Hereinafter, a method for detecting a nucleic acid using the nucleic acid detection structure will be described.

The sample from which the nucleic acid is to be detected may include mixing the sample with a cell lysis buffer before application to the sample pad. When the sample is mixed with the cell lysis buffer, the nucleic acid material present in the cell may be eluted, thereby increasing the amount of detectable nucleic acid material in the sample.

The cell lysates mixed with a cell lysis buffer (pH of 8.0 to 9.0) containing 5 mM to 80 mM of Tris-HCl, 5 mM to 50 mM of potassium chloride, 1 mM to 30 mM of magnesium sulfate, 5 mM to 50 mM of ammonium sulfate, 0.01 mg/ml to 0.1 mg/ml of protease, and 0.01 w/w% to 0.2 w/w% of TritonX-100 or Tween20, may be added dropwise to the sample pad, and the addition buffer may be added. The addition buffer serves to purify the nucleic acid material while allowing the nucleic acid material to move to the reaction pad. The addition buffer serves to purify the nucleic acid material while allowing the nucleic acid material to move to a conversion pad. Since the addition buffer contains an appropriate amount of the buffer component, it is possible to prevent precipitation of protein or nucleic acid material due to a rapid change in salinity or pH that may occur when distilled water is added. Each pad of a nucleic acid detection structure according to the present disclosure is made of a porous material having small pores so that the nucleic acid may be purified while moving laterally. Therefore, when the protein or nucleic acid material forms a complex salt or aggregates to block the pores, the sample movement speed may be reduced and the yield of the nucleic acid material may be degraded.

The addition buffer may be, for example, an isothermal buffer or a phosphate buffer (50 mM Na₂HPO₄, pH 7.2) containing 5 mM to 80 mM of Tris-HCl, 20 mM to 70 mM of potassium chloride, 0.5 mM to 5 mM of magnesium sulfate, 1 mM to 30 mM of ammonium sulfate, and 0.01 w/w% to 0.2 w/w% of Tween® 20 or TritonX-100 and having an acidity of pH 8.0 to 9.0. More specifically, the isothermal buffer may contain 20 mM of Tris-HCl, 10 mM of (NH₄)₂SO₄, 50 mM of KC1, 2 mM of MgSO₄, and 0.1% of Tween® 20, and may have an acidity of pH 8.8. The addition buffer may not contain protease, glycerol, or a reducing agent.

In order for the nucleic acid amplification to occur smoothly, the heating pad below the reaction pad may be heated to 60 to 65° C. and allowed to stand for 20 minutes to 1 hour, preferably 20 minutes to 30 minutes while maintaining that temperature.

A cause of a disease that may be diagnosed using the multi-nucleic acid detection structure is a specific gene, and the specific gene may be used as an indicator of the disease. The diseases include, for example, metabolic diseases such as obesity, hypertension, diabetes, genetic diseases, cancer, etc. Viruses or bacteria are related to infectious diseases, and bacteria that may cause bacterial infections include, but are not limited to, bacteria, protozoa, parasites, fungi, etc.

### Advantageous Effects

Using the nucleic acid extraction and amplification method according to the present disclosure, sample preparation, nucleic acid amplification and detection may be performed at a time by one application of the sample, without separately performing each process. Since each step is not separately performed, the entire process is simplified, and various samples or devices are not required, and it may be easily performed without related engineers.

In addition, the method according to the present disclosure is not limited by location because it does not require complex devices to perform each step, is easy to distribute, and is economical because it is possible to detect a large number of nucleic acids by applying a single sample.

### BRIEF DESCRIPTION OF DRAWINGS

FIG. 1 is an illustration of an overall structure of a multi-nucleic acid detection structure according to the present disclosure.

FIG. 2 is a schematic diagram of the principle of obtaining a target nucleic acid on the detection pad 160.

FIG. 3 is a side structural view of a portion of the multi-nucleic acid detection structure according to the present disclosure.

FIG. 4 is a perspective view of a portion of the multi-nucleic acid detection structure according to the present disclosure.

FIG. 5 is an enlarged structural diagram of the detection pad 160.

FIG. 6 is an illustration showing the appearance of the multi-nucleic acid detection structure according to the present disclosure surrounded by a case.

FIG. 7 is an illustration showing a result of detecting SARS-CoV-2 from a blood sample.

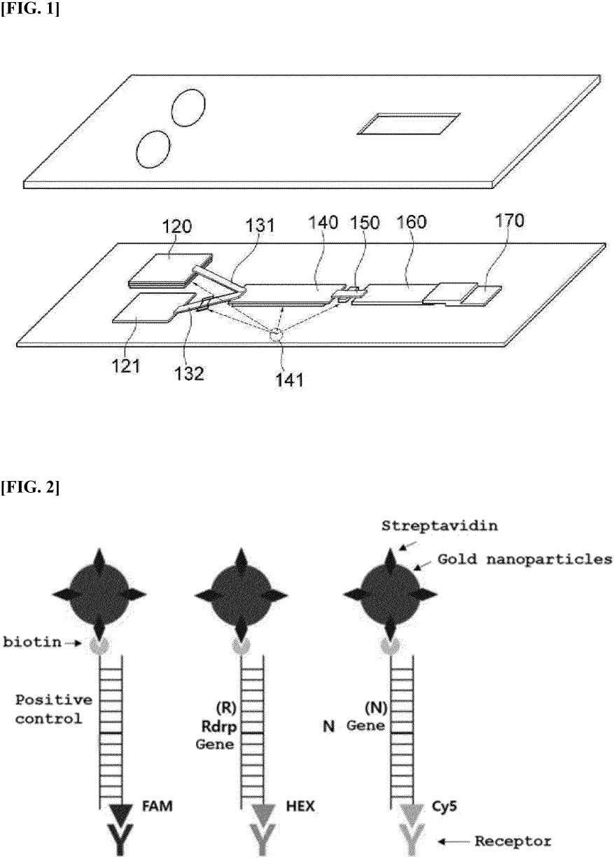

### BEST MODE

Hereinafter, the present disclosure will be described in more detail with reference to the following examples, but these are only for explaining the present disclosure, and the scope of the present disclosure is not limited in any way by these examples.

### Preparation Example 1. Preparation of Lab-On-Paper Nucleic Acid Detection Structure

In order to prepare the Lab-on-paper nucleic acid detection structure according to the present disclosure, the sample pad 120 was made of 0.5 µm polysulfone, the first connection pad 131 was made of 0.01 µm cellulose, the reaction pad 140 was made of 0.005 µm cellulose acetate, the second connection pad 150 was made of 0.05 µm cellulose, the detection pad 160 was made of 0.005 µm nitrocellulose, and the absorbent pad 170 was made of 0.5 µm glass fiber materials

The reaction pad 140 was prepared by overlapping a cellulose acetate membrane to make a pad, immersing the pad in a solution containing 45 mM of sucrose, 0.005 w/w% of TritonX-100, and 0.2 w/w% of glycerol, and then drying the pad. And, a well was formed in the pad with a fine drill, and a hydrogel layer including a primer set was formed on the bottom of the well. To form the hydrogel layer, first, 20% v/v of UV-photocrosslinkable poly(ethylene glycol) diacrylate (PEGDA, Sigma-Aldrich, MW700), 40% v/v of poly(ethylene glycol) (PEG, Sigma-Aldrich, MW600) and 5% v/v of 2-hydroxy-2-methylpropiophenone (Sigma-Aldrich) as a photoinitiator, and 35% of buffer (PBS buffer, pH 7.5) were mixed based on the total volume of the hydrogel solution, and each primer set was mixed thereto to prepare a hydrogel solution. The poly(ethylene glycol) is preferably included in order to increase porosity of the hydrogel microparticles. Then, the hydrogel solution was applied to the inner surface of each well of the reaction pad and exposed to UV for 1 minute (360 nm wavelength, 35 mJ/cm²) to form a hydrogel coating layer.

Next, dNTP (1.4 mM, dATP, dCTP, dGTP, and dTTP), powder containing loop-mediated isothermal amplification buffer (1X, 20 mM Tris-HCl, 10 mM (NH₄)₂SO₄, 50 mM KCl, 2 mM MgSO₄, and 0.1% Tween-20, pH 7.5), and Bst 3.0 DNA polymerase (320 U/ml) were applied to the surface of the reaction pad 140, and the reaction pad 140 was heated in an oven at about 38° C. for about 30 minutes and fixed.

The gold nanoparticles fixed to the second connection pad 150 were colloidal particles, and were prepared as follows. When a 0.1% HAuCl4 solution starts to boil while stirring and heating, 0.5% sodium citric acid solution was added to reduce the solution to make gold particles. The resulting gold particles were condensed by adding 1 mg each of streptavidin per 100 ml of the gold particle solution. The condensate was precipitated by centrifugation at 10.000 g, dissolved in physiological saline (PBS) containing 0.1% BSA, and stored so that an OD450 value became 10.

The second connection pad 150 was manufactured as follows. Specifically, several layers of cellulose membranes were prepared and cut, and soaked in a solution containing 0.4 M of Tris (pH 6.5), 0.2% of Tween-20, 1% of sodium caseinate, 0.1% of sodium azide, and 0.05% of Proclin 300. The prepared gold condensate was prepared by dialysis into a solution having the same composition as the solution. Then, the cellulose membrane was treated with the dialyzed gold condensate and dried to complete the second connection pad 150.

The detection pad 160 was designated by stamping a detection zone with polyethylene phthalate ink, stamped again with a solution containing an antibody capable of binding to fluorescent markers such as FAM, HEX, and Cy5 thereon, and then an N-hydroxysulfosuccinimide (NHS) solution was applied and reacted to immobilize the antibodies.

A portion of the second connection pad 150 in contact with the reaction pad was coated with a 5% low-melting-point agarose (Lonza, NuSieve GTG Agarose) solution.

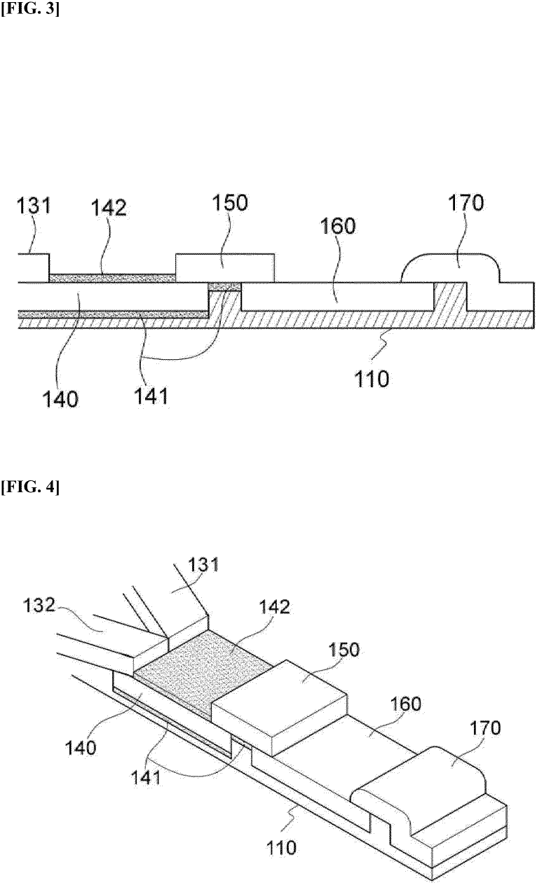

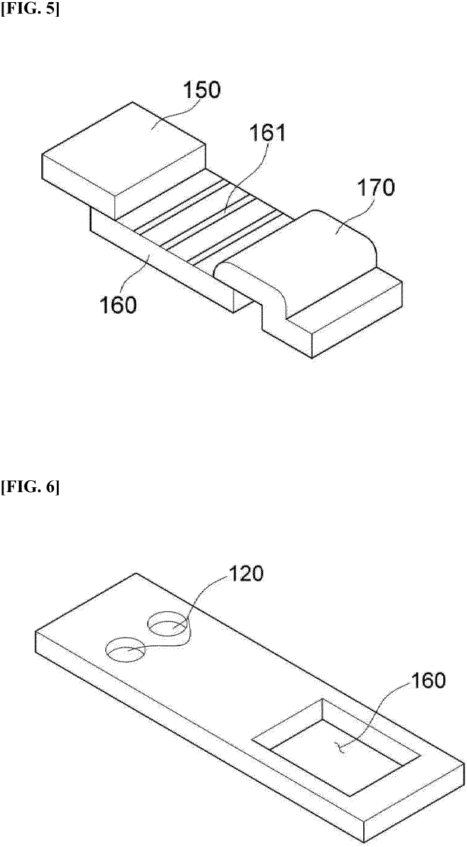

Then, the components of each structure were arranged as shown in FIG. 3, wherein a copper plate to which a hot wire is connected to a heating pad 141 was disposed at the lower portion of the sample pad 120, the reaction pad 140, and the second connection pad 150, and a polyacrylic film was disposed on the reaction pad as a blocking pad 142.

### Experimental Example 1 Virus Detection Using Blood Samples

It was possible to easily measure whether it was infected with a virus such as SARS-CoV-2 by extracting nucleic acids from blood samples, amplifying extracted nucleic acids, and measuring fluorescence, by using the Lab-on-paper nucleic acid detection structure according to the present disclosure.

In order to detect SARS-CoV-2, a primer set capable of selectively binding to N protein gene specific for SARS-CoV-2 or Rdrp gene may be used. When such a primer set was used, either the forward primer or the backward primer of each set was, for example, in a form bound to FAM, HEX, or Cy5, and the other primer was bound to biotin. When a target nucleic acid capable of specifically binding to the primer set is present in the sample, biotin amplified in the reaction pad 140 and bound to the amplified nucleic acid while passing through the second connection pad 150 is specifically bound to streptavidin of the gold nanoparticles. The amplified nucleic acid bound to the gold nanoparticles by the lateral flow moved to the detection pad 160, and the detector bound to the other side of the amplified nucleic acid binds to a receptor capable of specifically binding to FAM, HEX, or Cy5 fixed to the detection pad 160 to change the color of the detection area of the detection pad to pink.

The principle of binding of the target nucleic acid to the detection pad is schematically shown in FIG. 2.

After an additional detection zone on the detection pad in order to increase reliability was formed, a primer set that selectively binds to a gene specific for the virus with high potentialfor cross-detection with SARS-CoV-2 was introduced and detected together as a negative control.

### Experimental Example 2 Nucleic Acid Extraction and Amplification Using Blood Samples

### (1) Preparation of Test Samples and Paper Chips

It was confirmed whether nucleic acids could be extracted from blood samples using the Lab-on-paper nucleic acid detection structure according to the present disclosure and the extracted nucleic acids could be amplified to exhibit fluorescence.

First, human blood as whole blood was purchased from Innovative Research (IWB1K2E10ML, USA) and prepared, and 18S rRNA primer as a positive control was purchased from Tocris (#7325, USA). It was confirmed through the data sheet that the whole blood used was not infected with any virus or bacteria. One test sample was prepared by mixing 1 µl of 0.1 pg/µl SARS-CoV-2 positive control from siTOOLs Biotech with 100 µl of human blood.

A primer set for detection of SARS-CoV-2 mixed in blood is shown in Table 1 below.

TABLE 1

| Gene | Primer type | Sequence |
|---|---|---|
| N gene of SARS-CoV-2 | F3 | 5′-ACCGAAGAGCTACCAGACGA-3′ |
| | B3 | 5′-CTGCGTAGAAGCCTTTTGGC-3′ |
| | FIP | 5′-TCCAGCTTCTGGCCCAGTTCCTTTTTATTCGTGGTGGTGACGGTAA-3′ |
| | BIP | 5′-TATGGGTTGCAACTGAGGGAGCCTTTTTTCATTGTTAGCAGGATTGCGGG-3′ |
| | FL | 5′-ACCATCTTGGACTGAGATCTTTC-3′ |
| | BL | 5′-TACACCAAAAGATCACATTGGCA-3′) |

∗F3, forward primer; B3, backward primer; FIP, forward inner primer; BIP, backward inner primer; FL, forward loop primer; BL, backward loop primer

In each of the primer sets, a detector was bound to the 5′ end of the F3 primer, and biotin was bound to the 5′ end of the B3 primer. FAM for Human 18 s RNA (A), which is a positive control, and Cy5 for N gene (B) were introduced as a detector.

### (2) Nucleic Acid Detection From Samples

50 µl of each of the test sample and the negative control sample not mixed with SARS-CoV-2 was taken, 50 µl of lysis buffer (20 mM Tris·HCl (pH 8.8), 15 mM MgSO₄, 15 mM KCl, 15 mM (NH₄)₂SO₄, 0.1 w/w% Tween20, 0.05 mg/ml protease (Protenase K)) was added thereto, the resultant was lightly tapped, and then incubated at room temperature for about 5 minutes. Then, the resultant was slowly added dropwise within 5 minutes to the sample pad 120 of the nucleic acid detection structure manufactured in Preparation Example 1 while the heating pad 141 below the sample pad 120 was operated at 60° C., 250 µl of the addition buffer (20 mM Tris-HCl, 10 mM (NH₄)₂SO₄, 50 mM KCl, 2 mM MgSO₄, 0.1% Tween® 20, pH 8.8) was slowly added dropwise for about 2 minutes, and then the heating pad below the reaction pad was heated to 60° C. to react for 30 minutes. Thereafter, the heating pad below the second connection pad was heated to 65° C., 250 µl of the addition buffer was slowly added dropwise to the sample pad for 2 minutes, and the color change of the detection zone displayed on the detection pad was observed.

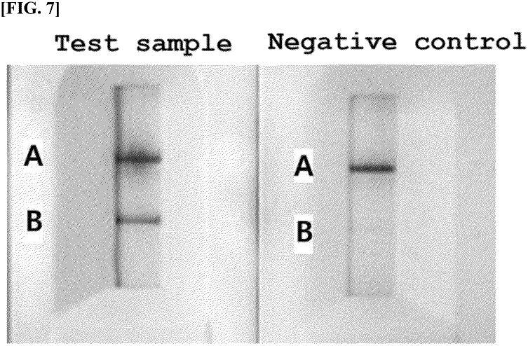

As a result, from FIG. 7 it was confirmed that the structure worked normally by confirming a positive control (A) band in both the test sample and the negative control. Also, it is was confirmed that SARS-CoV-2 (B) was detected in the test sample, whereas only the positive control (A) was observed in the negative control sample, so that the system according to the present disclosure may be used to diagnose whether a number of diseases, viral or bacterial infections are present.

1. A multi-nucleic acid detection structure, comprising:
a sample pad for receiving a biological sample;
a first connection pad disposed on the sample pad and connecting the sample pad and a reaction pad;
a reaction pad disposed below the first connection pad, including a primer capable of specifically binding to a target nucleic acid and a reagent for loop-mediated isothermal amplification (LAMP), and in which loop-mediated isothermal amplification occurs;
a blocking pad disposed on the reaction pad and configured to maintain a reaction temperature and block evaporation of the sample;
a second connection pad disposed on the reaction pad and having gold nanoparticles fixed thereon;
a detection pad disposed below the second connection pad and configured to obtain a target nucleic acid amplified from the loop-mediated isothermal amplification reactant bound to the gold nanoparticles;
an absorbent pad disposed laterally of the detection pad and absorbing the remaining sample; and
a heating pad disposed below the sample pad, the reaction pad, and the second connection pad.

2. The multi-nucleic acid detection structure of claim 1, wherein the sample pad and the first connection pad; the reaction pad; the second connection pad; the detection pad; and the absorbent pad are sequentially disposed laterally at least in partial contact with each other.

3. The multi-nucleic acid detection structure of claim 1, wherein the detection pad includes a plurality of divided detection zones.

4. The multi-nucleic acid detection structure of claim 1, wherein the gold nanoparticles include streptavidin on their surface.

5. The multi-nucleic acid detection structure of claim 1, wherein the reaction pad includes a set of forward and backward primers, and one of the forward and backward primers is biotin-bound, and the other primer is labeled with one or more fluorescent markers selected from the group consisting of Cy3, Cy5, TAMRA, TEX, TYE, HEX, FAM, TET, JOE, MAX, ROX, VIC, Cy3.5, Texas Red, Cy5.5, TYE, BHQ, Iowa Black RQ, and IRDye.

6. The multi-nucleic acid detection structure of claim 1, wherein the sample applied to the sample pad moves laterally to the absorbent pad.

7. The multi-nucleic acid detection structure of claim 1, wherein the sample is mixed with a cell lysis buffer containing 5 mM to 80 mM of Tris-HCl (pH 8.0 to 9.0), 5 mM to 50 mM of potassium chloride, 1 mM to 30 mM of magnesium sulfate, 5 mM to 50 mM of ammonium sulfate, 0.01 mg/ml to 0.1 mg/ml of protease, and 0.01 w/w% to 0.2 w/w% of TritonX-100 or Tween20 as a surfactant.

8. A kit for diagnosing a disease, viral or bacterial infection, comprising the multi-nucleic acid detection structure of claim 1.

9. A method of providing information for diagnosing a disease, viral or bacterial infection, the method comprising:
applying a biological sample to a sample pad of the multi-nucleic acid detection structure of claim 1, and amplifying a target nucleic acid; and
detecting the nucleic acid amplification product on a detection pad.

10. The method of claim 9, further comprising adding an additional buffer dropwise to the sample pad after applying the biological sample.
